(19) United States
(12) Patent Application Publication
(10) Pub. No.: US 2020/0155301 A1
(43) Pub. Date: May 21, 2020

(54) MOLDS FOR FORMING DERMAL ALLOGRAFT IMPLANTS AND DERMAL ALLOGRAFT IMPLANTS FORMED FROM SAME

(71) Applicants: ARTHROSURFACE, INC., Franklin, MA (US); COMMUNITY BLOOD CENTER dba COMMUNITY TISSUE SERVICES, Dayton, OH (US)

(72) Inventors: Nikhil T. JAWRANI, Bellingham, MA (US); Steven W. EK, Bolton, MA (US); Shawn A. HUNTER, Springboro, OH (US); Stephen W. GILL, Columbus, OH (US); Gregory S. LUCOUS, Brookville, OH (US); Ana Ysabel RIOJA, Dayton, OH (US)

(21) Appl. No.: 16/506,702

(22) Filed: Jul. 9, 2019

Related U.S. Application Data
(60) Provisional application No. 62/695,337, filed on Jul. 9, 2018.

(51) Int. Cl. A61F 2/10

(52) U.S. Cl. CPC A61F 2/105; A61F 2230/0006; A61B 17/0401; A61F 2230/0091; A61F 2220/0016; A61F 2240/004

(57) ABSTRACT

The present disclosure includes devices, apparatuses, and methods for forming lyophilized soft-tissue allografts for the correction of skeletal impairments (e.g., misalignments, arthritis, etc.), and soft-tissue allograft implants formed from the same.

### CROSS REFERENCE TO RELATED APPLICATIONS

This application claims the benefit of priority to U.S. Provisional Patent Application Ser. No. 62/695,337, filed Jul. 9, 2018, hereby incorporated by reference in its entirety.

### TECHNICAL FIELD

The present application relates generally to surgical devices for forming soft-tissue allograft implants (e.g., dermal allograft) for the correction of skeletal impairments (e.g., misalignment, arthritis, etc.), and more particularly, but not by way of limitation, to molds for forming lyophilized soft-tissue allograft implants.

### BACKGROUND

Examples of devices and methods that can be used for forming soft-tissue allografts, and allografts formed from the same, are disclosed in U.S. Pat. No. 7,189,263 (the '263 Patent). Another example of a surgical tool for forming implants is disclosed in U.S. Pat. No. 7,618,451 (the '451 Patent). An example of a lyophilized bone implant for correction of skeletal misalignment is disclosed in U.S. Pat. No. 6,162,258 (the '258 Patent). An example of a method for molding an implant is disclosed in U.S. Patent Publication No. 2013/0110470 (the '470 Publication).

One example of a skeletal impairment condition is a fallen arch or “flatfoot.” The condition involves a deformity in which the arches of the foot collapse, resulting in the entire sole of the foot being in complete or nearly complete contact with the ground. This may eventually cause other biomechanical issues with the physiology of the foot that, in turn, may adversely affect other parts of the body. The “flatfoot” condition occurs when the head of the talus bone is displaced medially and distally from the navicular bone, which in turn causes lateral misalignment throughout the foot as the talus and navicular bones tend to move outward. Furthermore, there is a change in relative alignment in the subtalar joint that occurs at the meeting point between the talus bone and the calcaneus bone such that the canal, which should naturally occur between the talus and calcaneus bones, is depressed. This canal is commonly referred to as the sinus tarsi. The misalignment of the talus and calcaneus bones eventually leads to misalignment of other bones in the foot and leg.

Another example of a skeletal impairment condition is osteoarthritis of the carpometacarpal (CMC) joint. The CMC joint is where the saddle-shaped trapezium bone articulates with the first metacarpal bone. An osteoarthritic CMC joint can become painful enough to severely limit activities of daily life for a large portion of the population. While symptoms may be treated with physical therapy, rest and stabilization, or anti-inflammatory medications, surgical intervention may be clinically indicated if pain persists. Interpositional arthroplasty of the CMC joint is the most common surgical procedure to treat osteoarthritis of the CMC joint.

Joint misalignment from osteoarthritis or other bone conditions may require repairing a defect in a joint using soft-tissue allografts. Current methods for repair are not sufficiently accurate to reproduce the natural movement of the joint such as the CMC joint. Typically, surgical planning for a joint misalignment is performed based on two-dimensional (2D) x-rays. During the procedure, a surgeon visually examines a defect and attempts to form an implant by hand that conforms to and fills the defect. The surgeon forms the implant from a material such as the flexor carpi radialis (FCR) tendon and shapes the material by rolling it into a plug form.

Surgical intervention to treat osteoarthritis of the CMC joint begins with removal of a portion or all of the trapezium bone to create a void. To prevent complete collapse of the first metacarpal bone into the void created, a wire pin is used as a temporary stabilizer to align the base of the first metacarpal bone with the base of the index metacarpal. The FCR tendon is then harvested, rolled up and sutured to prevent unrolling, and is interposed between the base of the thumb metacarpal and the scaphoid, the space previously occupied by the trapezium bone. In some cases, an additional procedure called a suspensionplasty is performed, where another piece of tendon is used to tie the base of the thumb metacarpal to the base of the index metacarpal.

Performing joint repairs in the manner described above is tedious and time consuming. In addition, it is difficult to accurately form an implant by hand that conforms to a defect and fills the defect to provide a continuous surface with the surface surrounding the defect. As a result, the natural movement of the joint may not be reproduced, and in some instances, even further compromised.

Thus, there is a need for forming soft-tissue allograft implants with improved handling and load bearing, which are flexible and not brittle, and are compression resistant.

It is an object of the present disclosure to provide an effective tool for forming soft-tissue allograft implants in a variety of forms, including anatomically representative forms corresponding to an articular surface geometry of a patient's joint.

It is another object of the present disclosure to provide soft-tissue allograft implants with improved handling and load bearing, which are flexible and not brittle, and are compression resistant.

It is a further object of the present disclosure to provide surgeons with an easy and efficient way of forming a soft-tissue allograft for implantation into a bony space and with decreased tendency to deform over time.

### SUMMARY

This disclosure includes configurations of devices, apparatuses, and methods for forming lyophilized soft-tissue allograft implants (e.g., dermal allograft) for the correction of skeletal impairments (e.g., misalignment, arthritis, etc.), and lyophilized soft-tissue allograft implants formed from the same. Non-limiting examples of surgical procedures that benefit from the present disclosure include, but are not limited to: subtalar joint arthroplasty; carpometacarpal joint arthroplasty; lateral mid-foot interpositional arthroplasty (e.g., 4^{th}/5^{th} metatarsal-cuboid joint); ankle interpositional arthroplasty (e.g., tibiotalar joint); elbow interpositional arthroplasty (e.g., radio-capitellar joint); proximal femoral interpositional arthroplasty; and interphalangeal interpositional arthroplasty (e.g., proximal interphalangeal joints of the fingers).

For example, at least some of the present configurations include a mold for forming lyophilized allograft implants, comprising a body having a first side and a second side and defining a chamber for receiving an allograft tissue, the chamber having a periphery extending between and through the first side and the second side; a first end cap configured to be coupled to the first side of the body over the chamber, the first end cap having a body-facing side with a surface portion that is aligned with the chamber when the first end cap is coupled to the body, and an outer side defining a plurality of holes extending between and through the outer side and the body-facing side and in fluid communication with the surface portion of the first end cap; and a second end cap configured to be coupled to the second side of the body over the chamber, the second end cap having a body-facing side with a surface portion that is aligned with the chamber when the second end cap is coupled to the body, and an outer side defining a plurality of holes extending between and through the outer side and the body-facing side and in fluid communication with the surface portion of the second end cap. The mold can be formed from metal and/or plastic using standard manufacturing processes (e.g., machining, molding, etc.) or can be 3D printed. In some configurations, the mold can also be made to have an inherent porosity (via 3D printing) to aid in the lyophilization process. In some configurations, the inner surface of the chamber may be smooth or roughened to impart desired surface properties onto the formed allograft.

In this way, at least some configurations of the present molds can aid in forming lyophilized soft-tissue allografts in a way that has previously not been possible with prior art implant molds. For example, at least some configurations of the present molds can aid in forming anatomically representative forms corresponding to an articular surface geometry of a patient's joint, and/or soft-tissue allograft implants with improved handling and load bearing, which are flexible and not brittle, and are compression resistant such that the tendency for the soft-tissue allograft to deform over time is minimized.

Some configurations of the present molds include a body, having a first portion and a second portion, that defines the chamber when the first portion and the second portion are coupled together. In other configurations, the body can have a plurality of portions that define the chamber when the plurality of portions are coupled together. The plurality of portions may be coupled together, for example, via a living hinge, a snap fit, or other mateable configurations. In this way, removal of the lyophilized soft-tissue allograft formed within the chamber can be performed by uncoupling the first portion and the second portion (or, in the case of a body with a plurality of portions, uncoupling the plurality of portions) to mitigate damaging the lyophilized soft-tissue allograft structure formed within the chamber.

In some configurations of the present molds, the first end cap and the second end cap are coupled to the body to compress and form allograft tissue into a shape conforming to the chamber during lyophilization. In some configurations of the present molds, the chamber is cylindrical.

In some configurations of the present molds, the surface portion of the first end cap is convex and the surface portion of the second end cap is convex. In some configurations of the present molds, the surface portion of the first end cap is concave and the surface portion of the second end cap is concave. In some configurations of the present molds, the surface portion of the first end cap is convex and the surface portion of the second end cap is concave. In some configurations of the present molds, the surface portion of the first end cap is concave and the surface portion of the second end cap is convex. In this way, the ends of the soft-tissue allograft implants formed in the molds can at least better anatomically represent forms corresponding to an articular surface geometry of a patient's joint and/or improve handling and load bearing.

In some configurations of the present molds, the body defines a plurality of longitudinal grooves extending along the periphery of the chamber. In some configurations of the present molds, the body defines a plurality of annular grooves extending around the periphery of the chamber. In some configurations of the present molds, the body defines one or more helical grooves extending around the periphery of the chamber.

In some configurations of the present molds, the periphery of the chamber corresponds to an articular surface geometry of a patient's joint. In some configurations of the present molds, the surface portion of the first end cap is convex and can have a shape corresponding to the articular surface geometry of the patient's joint, and the surface portion of the second end cap is convex and can have a shape corresponding to the articular surface geometry of the patient's joint. In some configurations of the present molds, the surface portion of the first end cap is concave and can have a shape corresponding to the articular surface geometry of the patient's joint, and the surface portion of the second end cap is concave and can have a shape corresponding to the articular surface geometry of the patient's joint.

In some configurations of the present molds, the body has a peripheral surface extending between the first side and the second side, and defining one or more suture passages extending through the peripheral surface and into the chamber.

Some configurations of the present dermal allografts include a body having a first end and a second end, the allograft including lyophilized and compressed tissue layers between the first end and the second end, where the body has a periphery corresponding to the shape of the chamber of any of the molds of presently disclosed.

In some configurations of the present dermal allografts, each of the first and second ends is concave. In some configurations of the present dermal allografts, each of the first and second ends is convex. In some configurations of the present dermal allografts, the first end is convex and the second end is concave. In some configurations of the present dermal allografts, the first end is concave and the second end is convex.

In some configurations of the present dermal allografts, the peripheral surface is cylindrical and has a circular cross-sectional shape.

In some configurations of the present dermal allografts, the body includes a plurality of longitudinal splines spaced around the periphery of the body.

In some configurations of the present dermal allografts, the body includes a plurality of annular splines around and spaced longitudinally along the periphery of the body.

In some configurations of the present dermal allografts, the body includes one or more helical splines and/or threads around and spaced longitudinally along the periphery of the body.

In some configurations of the present dermal allografts, the periphery of the body corresponds to an articular surface geometry of a patient's joint.

In some configurations of the present dermal allografts, the body defines a plurality of suture passages extending transversely through the body.

In some configurations of the present dermal allografts, the body defines a plurality of suture passages extending longitudinally through the first end and the second end.

In some configurations of the present dermal allografts, the body has a cross-sectional shape having a convex portion and a concave portion. In some configurations of the present dermal allografts, the cross-sectional shape has a first curved portion with a first radius, and second curved portion with a second radius that is larger than the first radius.

In some configurations of the present dermal allografts, the body defines one or more channels extending between and through the first end and second end.

In some configurations of the present dermal allografts, a fixation component is embedded in the body. In some configurations of the present dermal allografts, the fixation component is a suture anchor. In some configurations of the present dermal allografts, the fixation component is a bone screw.

Some implementations of the present methods for forming lyophilized soft-tissue allografts include (a) inserting a soft-tissue allograft into the chamber of any of the molds presently disclosed; (b) coupling the first end cap and second end cap to the body to compress the soft-tissue allograft; and (c) lyophilizing the soft-tissue allograft disposed within the chamber. In this way, at least some implementations of the present methods can form soft-tissue allograft implants that are resiliently compressible and flexible while remaining substantially as the formed shape.

In some implementations of the present methods, the soft-tissue allograft can be lyophilized one or more times prior to insertion into the chamber. In some implementations of the present methods, the soft-tissue allograft can be lyophilized anywhere from 10% to 90% prior to insertion into the chamber for further lyophilization. In some implementations of the present methods, the remaining lyophilization of the soft-tissue allograft takes place inside the chamber.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any configuration or implementation of the present devices, apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device, or structure that is configured in a certain way is configured in at least that way, but it can also be configured in other ways than those specifically described.

Any configuration or implementation of any of the present devices, apparatuses, kits, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the configurations described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configurations depicted in the figures.

FIG. 1A shows a perspective view of a configuration of a mold for forming concave-ended lyophilized soft-tissue allograft implants.

FIG. 1B shows a perspective view of a configuration of a body of the present molds having a first portion and a second portion coupled together.

FIG. 1C shows a cross-sectional view of the mold of FIG. 1A taken along a plane bisecting the first end cap, body, and second end cap.

FIG. 1D shows a top view of a configuration of the body of the mold of FIG. 1A and an end cap.

FIG. 2A shows a perspective view of a configuration of a concave-ended lyophilized soft-tissue allograft implant formed from the mold of FIG. 1A.

FIG. 2B shows a cross-sectional view of the lyophilized soft-tissue allograft implant of FIG. 2A taken along a plane longitudinally bisecting the implant.

FIG. 2C shows an end view of the lyophilized soft-tissue allograft implant of FIG. 2A.

FIG. 3A shows a perspective view of a configuration of a mold for forming convex-ended lyophilized soft-tissue allograft implants.

FIG. 3B shows a cross-sectional view of the mold of FIG. 3A taken along a plane bisecting the first end cap, body, and second end cap.

FIG. 3C shows a top view of a configuration of the body of the mold of FIG. 3A and an end cap.

FIG. 4A shows a perspective view of a configuration of a convex-ended lyophilized soft-tissue allograft implant formed from the mold of FIG. 3A.

FIG. 4B shows a cross-sectional view of the lyophilized soft-tissue allograft implant of FIG. 4A taken along a plane longitudinally bisecting the implant.

FIG. 4C shows an end view of the lyophilized soft-tissue allograft implant of FIG. 4A.

FIG. 5A shows a perspective view of a configuration of a lyophilized soft-tissue allograft implant with a convex end and a concave end.

FIG. 5B shows a cross-sectional view of the lyophilized soft-tissue allograft implant of FIG. 5A.

FIG. 5C shows an end view of the lyophilized soft-tissue allograft implant of FIG. 5A.

FIG. 6A shows a perspective view of a configuration of a mold for forming convex-ended lyophilized soft-tissue allograft implants with longitudinal splines.

FIG. 6B shows cross-sectional view of the mold of FIG. 6A taken along a plane bisecting the first end cap, body, and second end cap.

FIG. 6C shows a top view of a configuration of the body of the mold of FIG. 6A and an end cap.

FIG. 7A shows a perspective view of a configuration of a convex-ended lyophilized soft-tissue allograft implant with longitudinal splines.

FIG. 7B shows a side view of the lyophilized soft-tissue allograft implant of FIG. 7A.

FIG. 7C shows a cross-sectional view of the lyophilized soft-tissue allograft implant of FIG. 7A taken along a plane bisecting the implant.

FIG. 7D shows an end view of the lyophilized soft-tissue allograft implant of FIG. 7A.

FIG. 8A shows a perspective view of a configuration of a mold for forming convex-ended lyophilized soft-tissue allograft implants with annular splines.

FIG. 8B shows cross-sectional view of the mold of FIG. 8A taken along a plane bisecting the first end cap, body, and second end cap.

FIG. 8C shows a top view of a configuration of the body of the mold of FIG. 8A and an end cap.

FIG. 9A shows a perspective view of a configuration of a convex-ended lyophilized soft-tissue allograft implant with annular splines.

FIG. 9B shows a cross-sectional view of the lyophilized soft-tissue allograft implant of FIG. 9A taken along a plane bisecting the implant.

FIG. 9C shows an end view of the lyophilized soft-tissue allograft implant of FIG. 9A.

FIG. 10A shows a perspective view of a configuration of a mold for forming lyophilized soft-tissue allograft implants corresponding to an articular surface geometry of a patient's joint.

FIG. 10B shows a cross-sectional view of the mold of FIG. 10A taken along a plane bisecting the first end cap, body, and second end cap.

FIG. 10C shows a top view of a configuration of the body of the mold of FIG. 10A and an end cap.

FIG. 11A shows a perspective view of a configuration of a lyophilized soft-tissue allograft implant formed from the mold of FIG. 10A.

FIG. 11B shows a first side view of the lyophilized soft-tissue allograft implant of FIG. 11A.

FIG. 11C shows a second side view of the lyophilized soft-tissue allograft implant of FIG. 11A.

FIG. 11D shows an end view of the lyophilized soft-tissue allograft implant of FIG. 11A.

FIG. 12A shows a perspective view of a configuration of a mold for forming lyophilized soft-tissue allograft implants having suture passages extending transversely through the soft-tissue allograft implant body.

FIG. 12B shows a cross-sectional view of the mold of FIG. 12A taken along a plane bisecting the first end cap, body, and second end cap.

FIG. 12C shows a top view of a configuration of the body of the mold of FIG. 12A and an end cap.

FIG. 13A shows a perspective view of a configuration of a mold for forming lyophilized soft-tissue allograft implants corresponding to an articular surface geometry of a patient's joint and having a fixation component (e.g., sutures) embedded within tissue layers.

FIG. 13B shows a top view of a configuration of the body of the mold of FIG. 13A with sutures threaded through the suture passages and an end cap.

FIG. 14A shows a perspective view of a configuration of a lyophilized soft-tissue allograft implant formed from the mold of FIG. 12A and having suture passages extending transversely through the soft-tissue allograft implant body.

FIG. 14B shows a side view of the lyophilized soft-tissue allograft implant of FIG. 14A.

FIG. 14C shows an end view of the lyophilized soft-tissue allograft implant of FIG. 14A.

FIG. 15A shows a first perspective view of a configuration of a lyophilized soft-tissue allograft implant formed from the mold of FIG. 12A and having suture passages extending longitudinally through the first end and the second end of the soft-tissue allograft implant body.

FIG. 15B shows a second perspective view of the lyophilized soft-tissue allograft implant of FIG. 15A.

FIG. 15C shows an end view of the lyophilized soft-tissue allograft implant of FIG. 15A.

FIG. 16A shows an exploded perspective view of a configuration of a mold for forming concave-ended lyophilized soft-tissue allograft implants corresponding to an articular surface geometry of a patient's joint.

FIG. 16B shows cross-sectional view of the mold of FIG. 16A taken along a plane bisecting the first end cap, body, and second end cap.

FIG. 16C shows a top view of a configuration of the body of the mold of FIG. 16A and an end cap.

FIG. 17A shows a perspective view of a configuration of a lyophilized soft-tissue allograft implant having a cross-sectional shape with a convex portion and a concave portion.

FIG. 17B shows a top view of the lyophilized soft-tissue allograft implant of FIG. 17A having a first curved portion with first radius R₁ and a second curved portion with second radius R₂, where R₂ is greater than R₁.

FIG. 17C shows a cross-sectional view of the lyophilized soft-tissue allograft implant of FIG. 17A taken along a plane in the direction indicated by line A-A.

FIG. 17D shows a cross-sectional view of the lyophilized soft-tissue allograft implant of FIG. 17A taken along a plane in the direction indicated by line B-B.

FIG. 18A shows a perspective view of a configuration of a lyophilized soft-tissue allograft implant having one or more channels extending between and through the first end and second end of the body.

FIG. 18B shows a top view of the lyophilized soft-tissue allograft implant of FIG. 18A having a first curved portion with first radius R₁ and a second curved portion with second radius R₂, where R₂ is greater than R₁.

FIG. 18C shows a cross-sectional view of the lyophilized soft-tissue allograft implant of FIG. 18A taken along a plane in the direction indicated by line A-A.

FIG. 18D shows a cross-sectional view of the lyophilized soft-tissue allograft implant of FIG. 18A taken along a plane in the direction indicated by line B-B.

FIG. 19A shows a perspective view of a configuration of a lyophilized soft-tissue allograft implant having a fixation component (e.g., suture anchor, bone screw) is embedded in the body.

FIG. 19B shows a top view of the lyophilized soft-tissue allograft implant of FIG. 19A having a first curved portion with first radius R₁ and a second curved portion with second radius R₂, where R₂ is greater than R₁.

FIG. 19C shows a cross-sectional view of the lyophilized soft-tissue allograft implant of FIG. 19A taken along a plane in the direction indicated by line A-A.

FIG. 19D shows a cross-sectional view of the lyophilized soft-tissue allograft implant of FIG. 19A taken along a plane in the direction indicated by line B-B.

FIG. 20 shows a flow chart for depicting a process for forming a lyophilized soft-tissue allograft using a configuration of the presently disclosed molds.

### DESCRIPTION OF ILLUSTRATIVE EMBODIMENTS

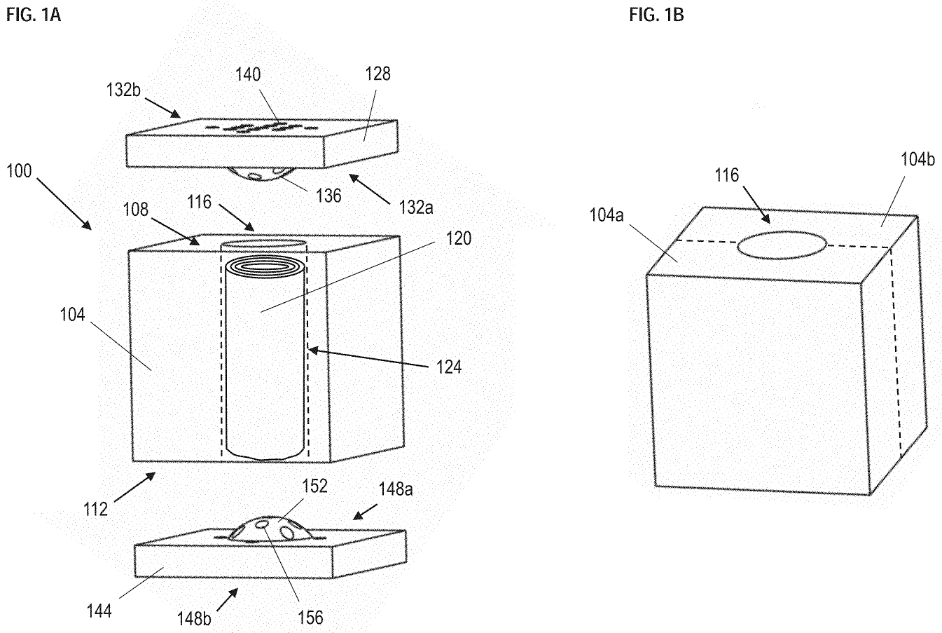

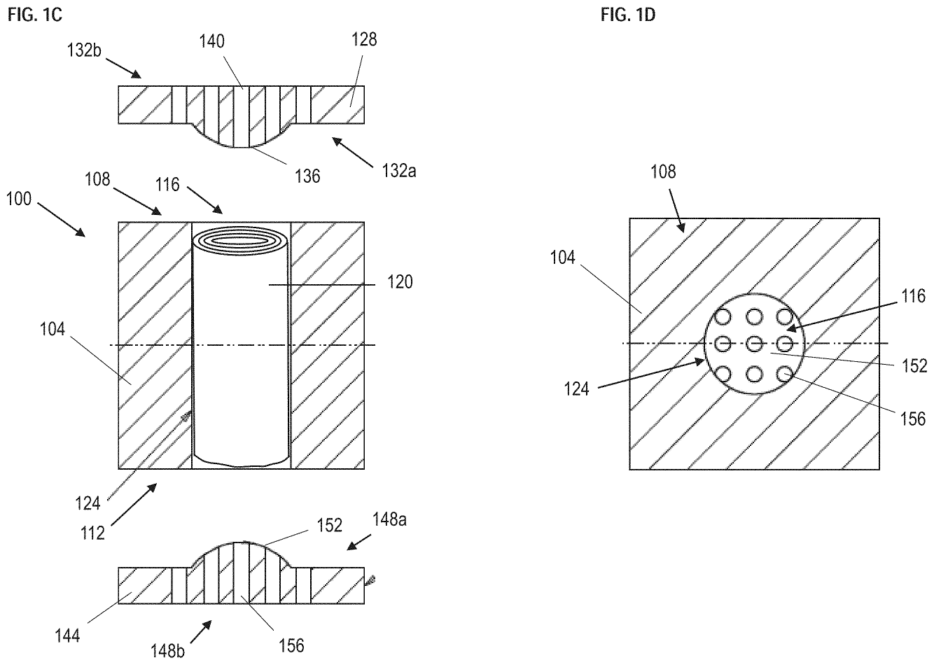

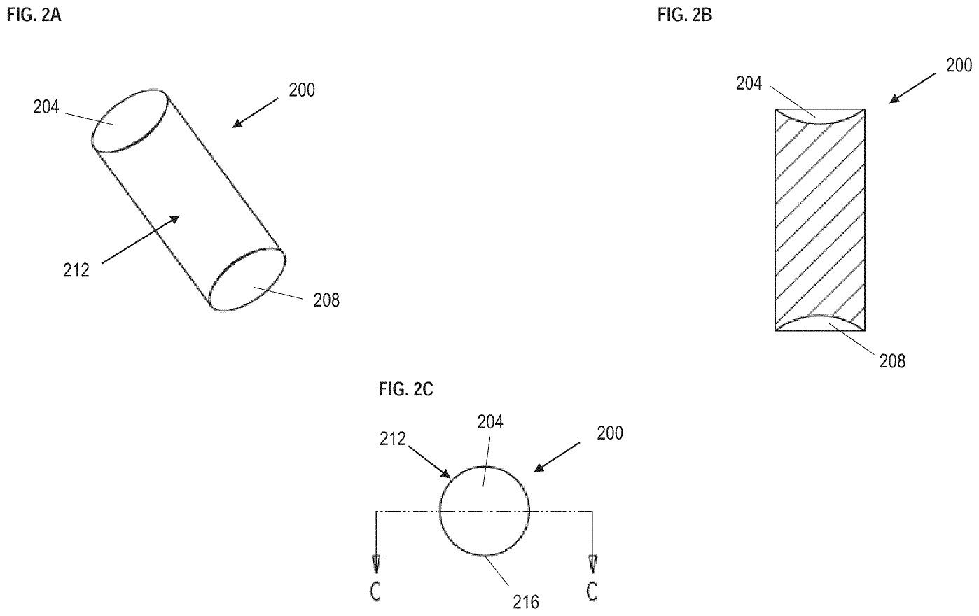

Referring now to the drawings, and more particularly to FIGS. 1A-2C, FIG. 1A shows a perspective view of a configuration of a mold for forming concave-ended lyophilized soft-tissue allograft implants; FIG. 1B shows a perspective view of a configuration of a body of the present molds having a first portion and a second portion coupled together; FIG. 1C shows a cross-sectional view of the mold of FIG. 1A taken along a plane bisecting the first end cap, body, and second end cap; FIG. 1D shows a top view of a configuration of the body of the mold of FIG. 1A and an end cap; FIG. 2A shows a perspective view of a configuration of a concave-ended lyophilized soft-tissue allograft implant formed from the mold of FIG. 1A; FIG. 2B shows a cross-sectional view of the lyophilized soft-tissue allograft implant of FIG. 2A taken along a plane longitudinally bisecting the implant; FIG. 2C shows an end view of the lyophilized soft-tissue allograft implant of FIG. 2A.

In some configurations, the mold 100 comprises a body 104 having a first side 108 and a second side 112 and defining a chamber 116 for receiving an allograft tissue 120 (e.g., dermal allograft), the chamber 116 having a periphery 124 extending between and through the first side 108 and the second side 112; a first end cap 128 configured to be coupled to the first side 108 of the body 104 over the chamber 116, the first end cap 128 having a body-facing side 132a with a surface portion 136 that is aligned with the chamber 116 when the first end cap 128 is coupled to the body 104, and an outer side 132b defining a plurality of holes 140 extending between and through the outer side 132b and the body-facing side 132a and in fluid communication with the surface portion 136 of the first end cap 128; and a second end cap 144 configured to be coupled to the second side 112 of the body 104 over the chamber 116, the second end cap 144 having a body-facing side 148a with a surface portion 152 that is aligned with the chamber 116 when the second end cap 144 is coupled to the body 104, and an outer side 148b defining a plurality of holes 156 extending between and through the outer side 148b and the body-facing side 148a and in fluid communication with the surface portion 152 of the second end cap 144. The plurality of holes 140 in first end cap 128 and the plurality of holes 156 in second end cap 144 can aid in the lyophilization of allograft tissue 120 by permitting excess moisture to escape during the freeze drying process. The mold 100 can be formed from metal and/or plastic using standard manufacturing processes (e.g., machining, molding, etc.) or can be 3D printed. In some configurations, the mold 100 can also be made to have an inherent porosity (via 3D printing) to aid in the lyophilization process. In some configurations, the inner surface of the chamber 116 may be smooth or roughened to impart desired surface properties onto the formed allograft tissue 120.

In some configurations, the first end cap 128 and the second end cap 144 are coupled to the body 104 to compress and form the allograft tissue 120 into a shape conforming to the chamber 116 during lyophilization. In some configurations, the chamber 116 is cylindrical.

In some configurations, the first end cap 128 can have a surface portion 136 that is convex. In some configurations, the first end cap 128 can have a surface portion 136 that is concave. In some configurations, the second end cap 144 can have a surface portion 152 that is convex. In some configurations, the second end cap 144 can have a surface portion 152 that is concave.

In some configurations, the body 104 can have a first portion 104a and a second portion 104b, and can define the chamber 116 when the first portion 104a and the second portion 104b are coupled together. In some configurations, the body 104 can have a plurality of portions that define the chamber when the plurality of portions are coupled together. The plurality of portions may be coupled together, for example, via a living hinge, a snap fit, or other mateable configuration. In this way, removal of the lyophilized soft-tissue allograft formed within the chamber can be performed by uncoupling the first portion 104a and the second portion 104b (or, in the case of a body with a plurality of portions, uncoupling the plurality of portions) to mitigate the risk of damaging the lyophilized soft-tissue allograft structure formed within the chamber 116.

In a particular configuration, such as the one shown in FIGS. 1A, 1C-1D, mold 100 can be configured to form a concave-ended lyophilized soft-tissue allograft 200, as shown in FIGS. 2A-2C. As best shown in FIG. 1C, mold 100 can have a surface portion 136 of the first end cap 128 that is convex and a surface portion 152 of the second end cap 144 that is convex. In this way, the lyophilized soft-tissue allograft 200 formed within the chamber 116 of body 104 can be formed such that each of the first and second ends (e.g., 204, 208) is concave, as best shown in FIGS. 2A-2B, and the peripheral surface 212 of the lyophilized soft-tissue allograft 200 is cylindrical and has a circular cross-sectional shape 216, as shown in FIG. 2C.

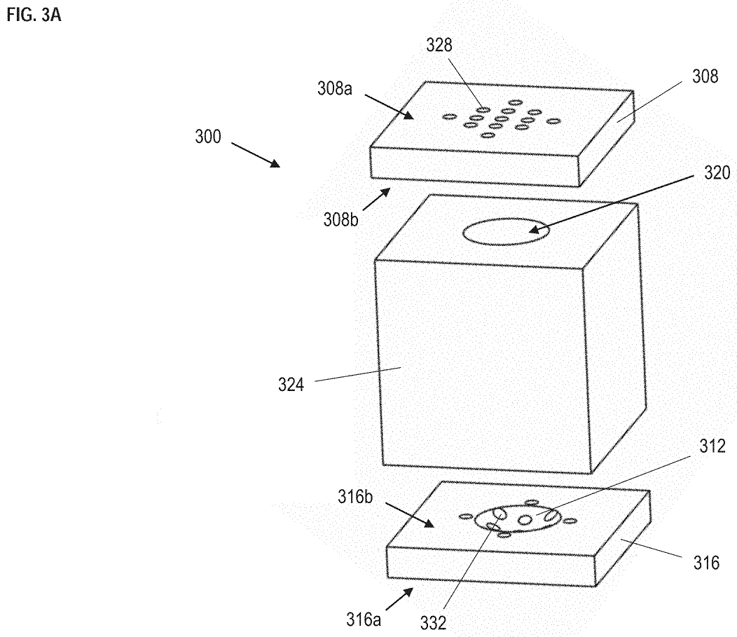

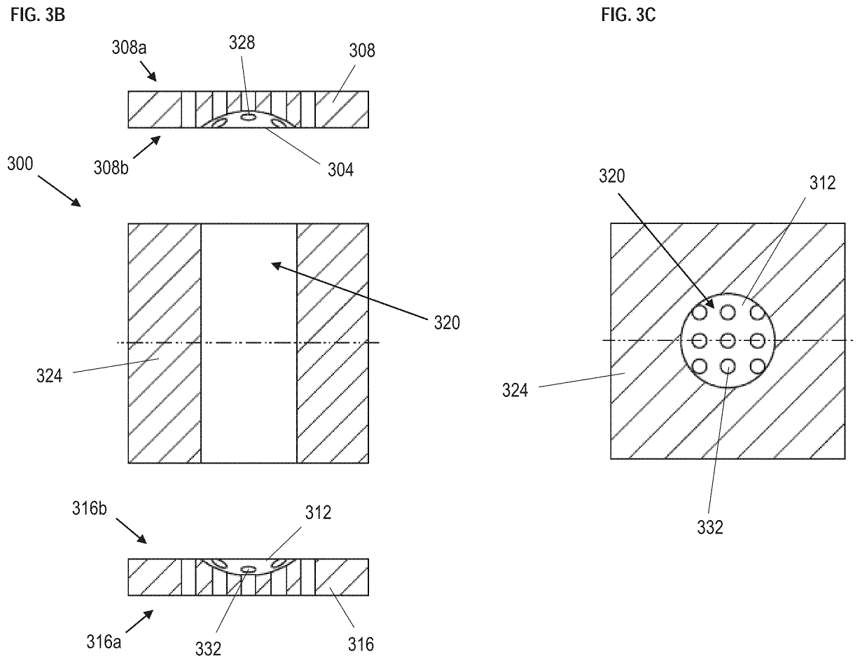

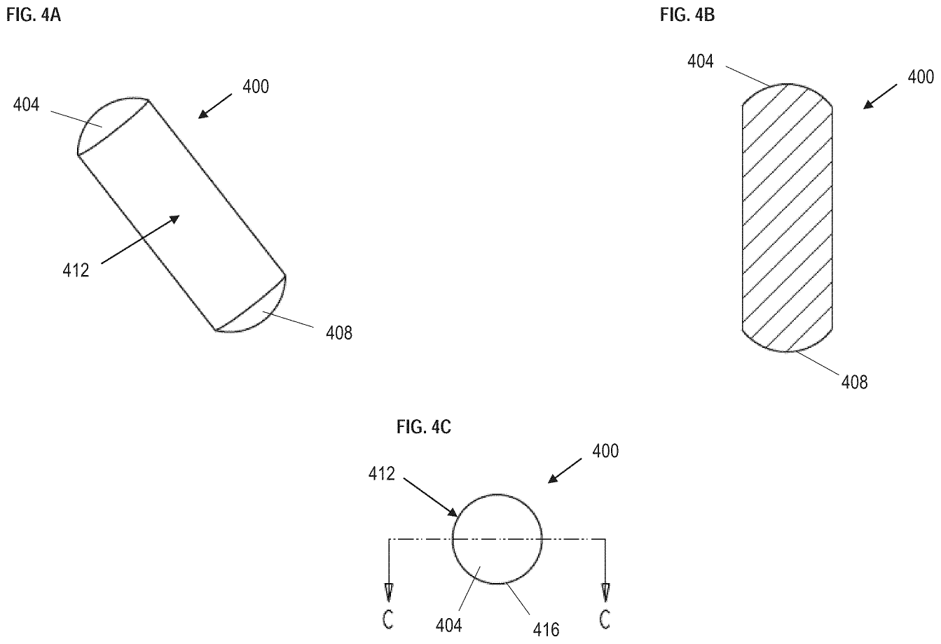

In some configurations, such as the one shown in FIGS. 3A-3C, mold 300 can be configured to form a convex-ended lyophilized soft-tissue allograft 400, as shown in FIGS. 4A-4C. As best shown in FIG. 3B, mold 300 can have a surface portion 304 of the first end cap 308 that is concave and a surface portion 312 of the second end cap 316 that is concave. In this way, the lyophilized soft-tissue allograft 400 formed within the chamber 320 of body 324 can be formed such that each of the first and second ends (e.g., 404, 408) of the lyophilized soft-tissue allograft 400 is convex, as best shown in FIGS. 4A-4B, and the peripheral surface 412 of the lyophilized soft-tissue allograft 400 is cylindrical and has a circular cross-sectional shape 416, as shown in FIG. 4C. The first and second end caps (308, 316) can have a plurality of holes (328, 332) extending between and through the outer sides (308a, 316a) and the body-facing sides (308b, 316b) of the first and second end caps (308, 316) and in fluid communication with the surface portions (304, 312) of the first end cap 308 and the second end cap 316 to aid in lyophilization.

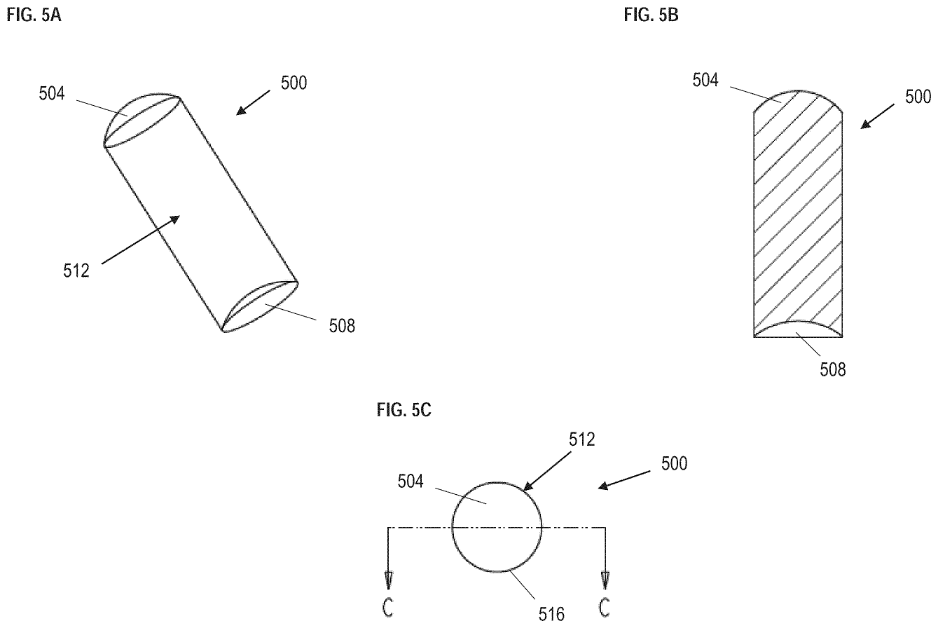

In some configurations, the mold (e.g., 100, 300) can be configured to form a lyophilized soft-tissue allograft 500 with a convex first end 504 and a concave second end 508, as shown in FIGS. 5A-5C, and the peripheral surface 512 of the lyophilized soft-tissue allograft 500 is cylindrical and has a circular cross-sectional shape 516. In this way, the mold (e.g., 100, 300) can have a surface portion (e.g., 304) of the first end cap (e.g., 308) that is concave and a surface portion (e.g., 152) of the second end cap (e.g., 144) that is convex.

In some configurations, mold (e.g., 100, 300) can be configured to form a lyophilized soft-tissue allograft with a concave first end and a convex second end, and the peripheral surface of the lyophilized soft-tissue allograft is cylindrical and has a circular cross-sectional shape. In this way, the mold (e.g., 100, 300) can have a surface portion of the first end cap that is convex and a surface portion of the second end cap that is concave.

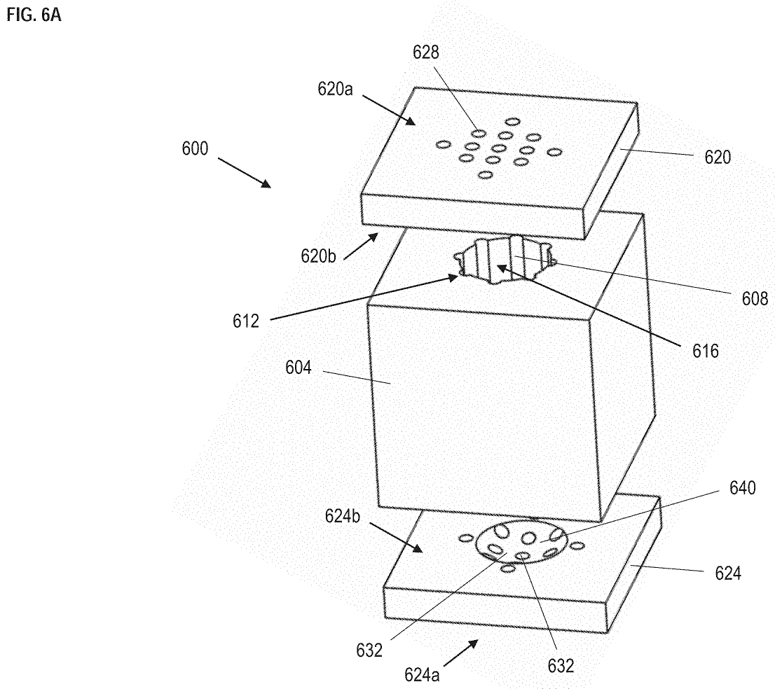

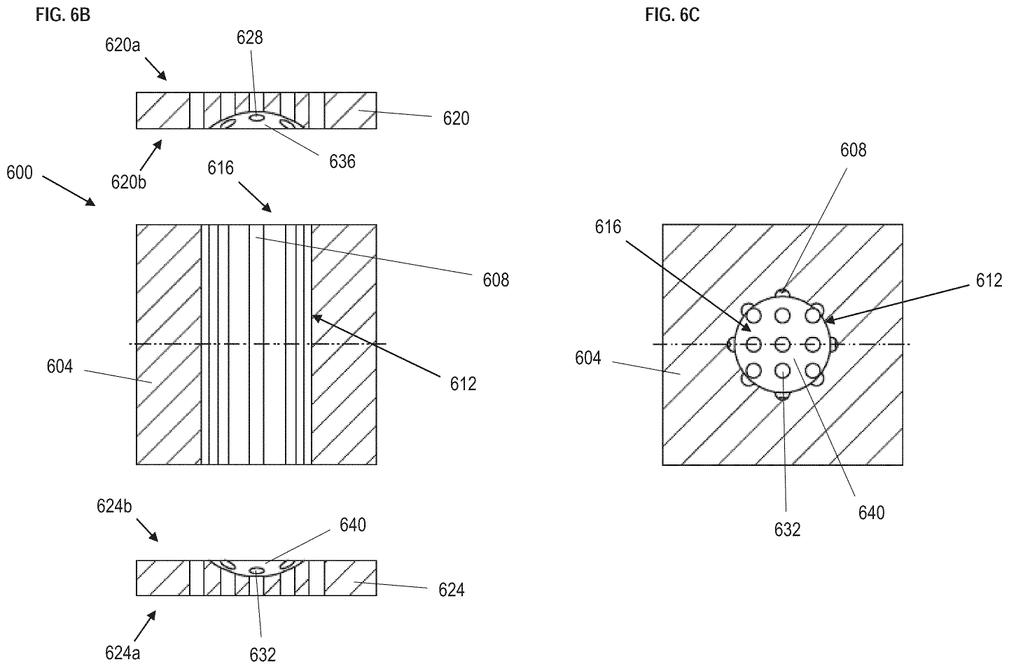

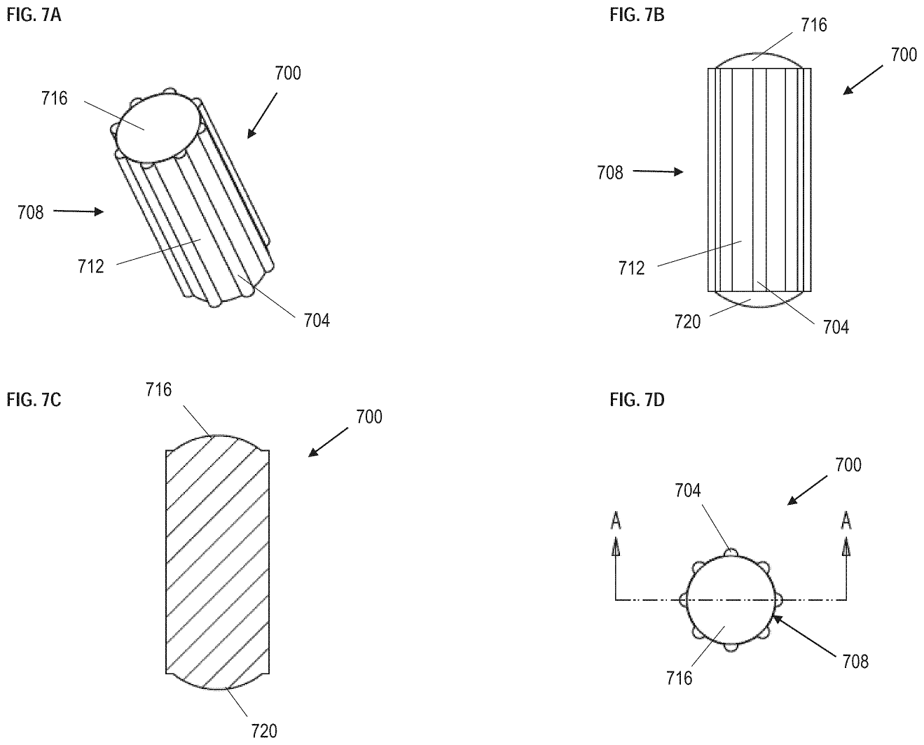

In a particular configuration, such as the one shown in FIGS. 6A-6C, mold 600 can be configured to form a lyophilized soft-tissue allograft 700 having a plurality of longitudinal splines 704 spaced around the periphery 708 of the body 712, as shown in FIGS. 7A-7D. In this configuration, as best shown in FIGS. 6A-6B, the body 604 of mold 600 defines a plurality of longitudinal grooves 608 extending along the periphery 612 of the chamber 616. The first and second end caps (620, 624) may be configured to have a surface portion that is either convex or concave, and a plurality of holes (628, 632) extending between and through the outer sides (620a, 624a) and the body-facing sides (620b, 624b) of the first and second end caps (620, 624) and in fluid communication with the surface portions (636, 640) of the first end cap 620 and the second end cap 624 to aid in lyophilization. As shown in FIG. 6B, the first end cap 620 and second end cap 624 each have a concave surface portion (636, 640 respectively), such that the lyophilized soft-tissue allograft 700 formed in the chamber 616 will have convex first and second ends (716, 720), as best shown in FIGS. 7B-7C.

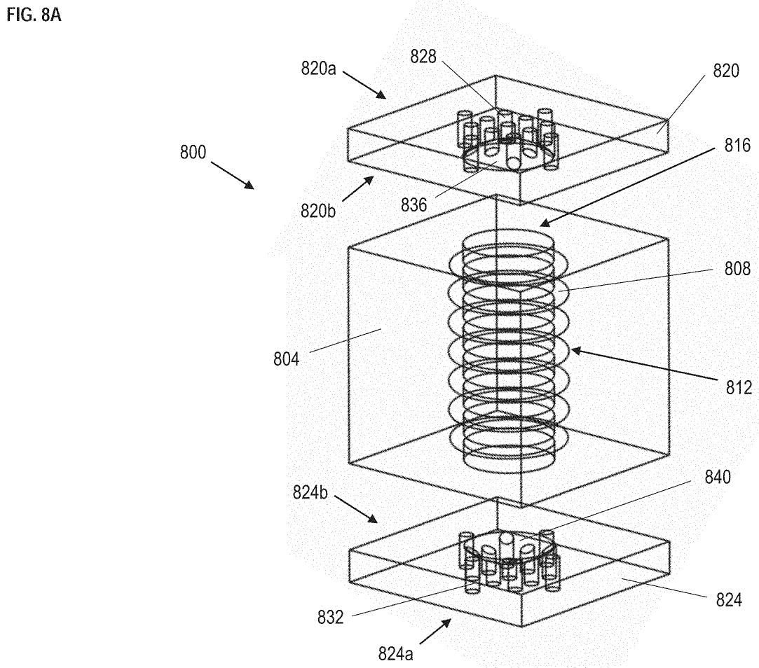

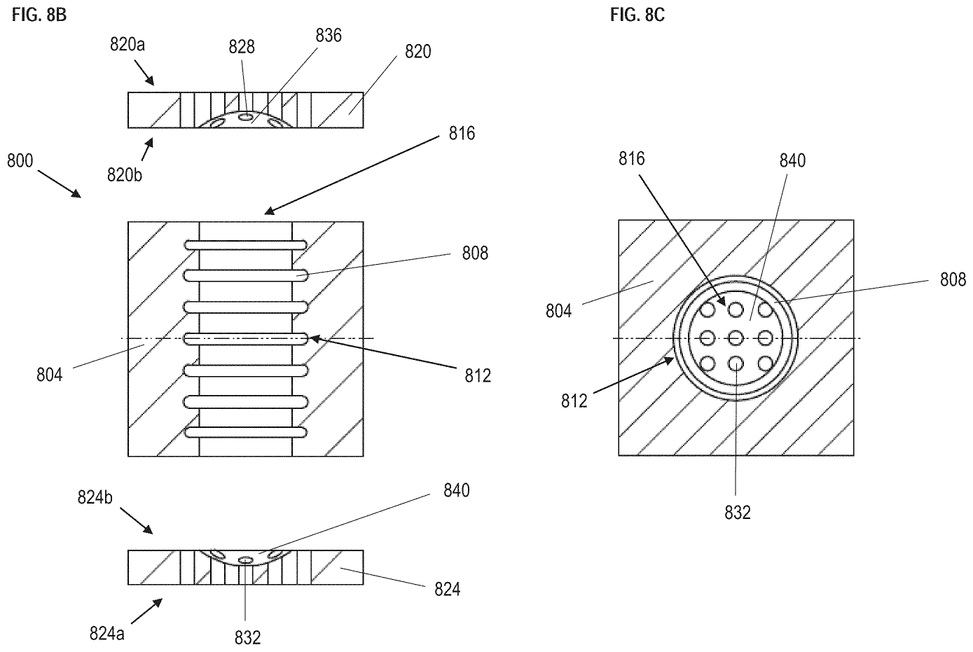

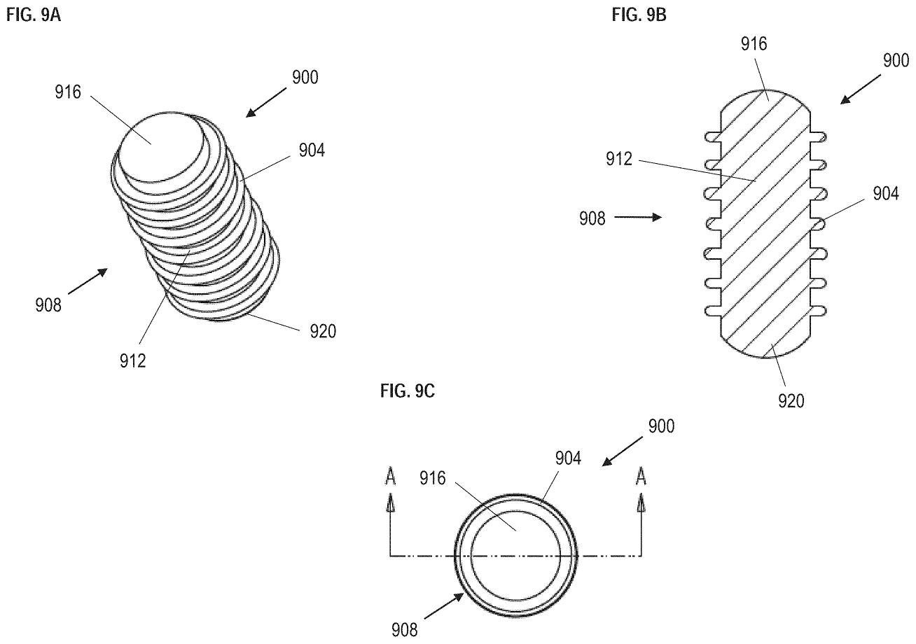

In a particular configuration, such as the one shown in FIGS. 8A-8C, mold 800 can be configured to form a lyophilized soft-tissue allograft 900 having a plurality of annular splines 904 around and spaced longitudinally along the periphery 908 of the body 912, as shown in FIGS. 9A-9C. In this configuration, as best shown in FIGS. 8A, 8B, the body 804 of mold 800 defines a plurality of annular grooves 808 extending around the periphery 812 of the chamber 816. The first and second end caps (820, 824 respectively) may be configured to have a surface portion that is either convex or concave, and a plurality of holes (828, 832) extending between and through the outer sides (820a, 824a) and the body-facing sides (820b, 824b) of the first and second end caps (820, 824) and in fluid communication with the surface portions (836, 840) of the first end cap 820 and the second end cap 824 to aid in lyophilization. As shown in FIGS. 8A, 8B, the first end cap 820 and second end cap 824 each have a concave surface portion (836, 840 respectively), such that the lyophilized soft-tissue allograft 900 formed in the chamber 816 will have convex first and second ends (916, 920), as best shown in FIGS. 9A, 9B.

In some configurations, the mold (e.g., 800) can be configured to form a lyophilized soft-tissue allograft having one or more helical splines and/or threads around and spaced longitudinally along the periphery of the body. In this configuration, the body (e.g., 804) of mold (e.g., 800) defines one or more helical grooves extending around the periphery of the chamber. The first and second end caps may be configured to have a surface portion that is either convex or concave, and a plurality of holes extending between and through the outer sides and the body-facing sides of the first and second end caps and in fluid communication with the surface portions of the first end cap and the second end cap to aid in lyophilization.

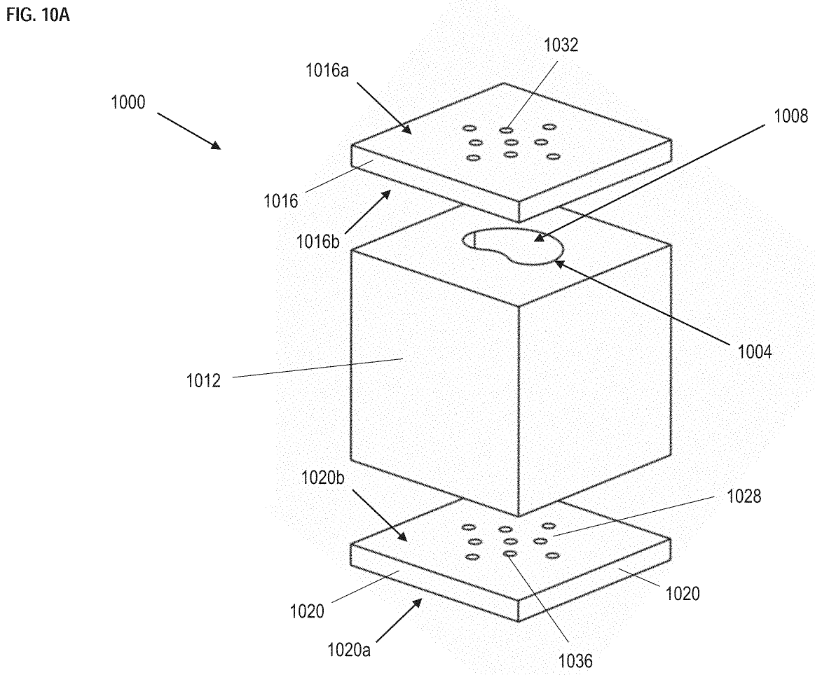

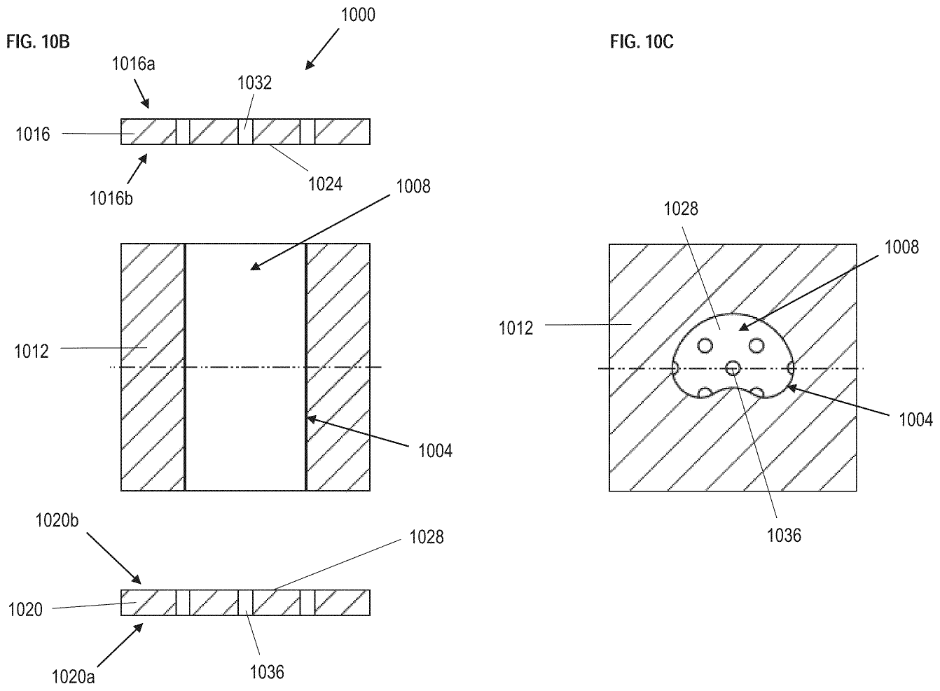

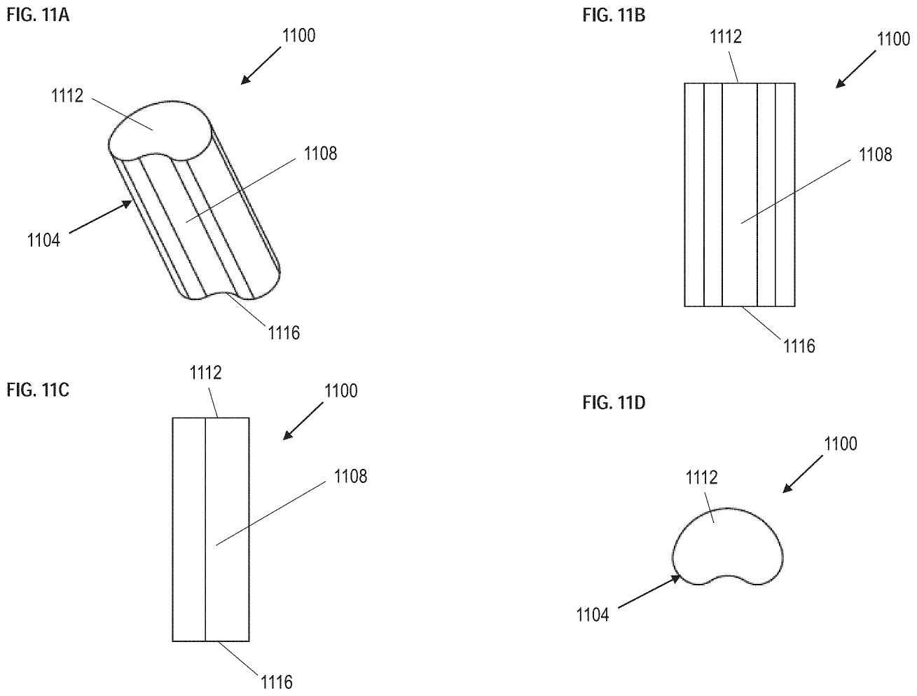

In a particular configuration, such as the one shown in FIGS. 10A-10C, mold 1000 can be configured to form a lyophilized soft-tissue allograft 1100 where the periphery 1104 of the allograft body 1108 corresponds to an articular surface geometry of a patient's joint, as best shown in FIGS. 11A, 11D. In this configuration, as best shown in FIGS. 10A, 10C, the periphery 1004 of the chamber 1008 of the body 1012 corresponds to an articular surface geometry of a patient's joint. The first and second end caps (1016, 1020 respectively) may be configured to have a convex or concave surface portion that corresponds to an articular surface geometry of the patient's joint. As shown in FIGS. 10A, 10B, the first end cap 1016 and second end cap 1020 each have a flat surface portion (1024, 1028 respectively), such that the lyophilized soft-tissue allograft 1100 formed in the chamber 1008 will have flat ends (1112, 1116), as best shown in FIGS. 11A-11C, and a plurality of holes (1032, 1036) extending between and through the outer sides (1016a, 1020a) and the body-facing sides (1016b, 1020b) of the first and second end caps (1016, 1020) and in fluid communication with the surface portions (1024, 1028) of the first end cap 1016 and the second end cap 1020 to aid in lyophilization.

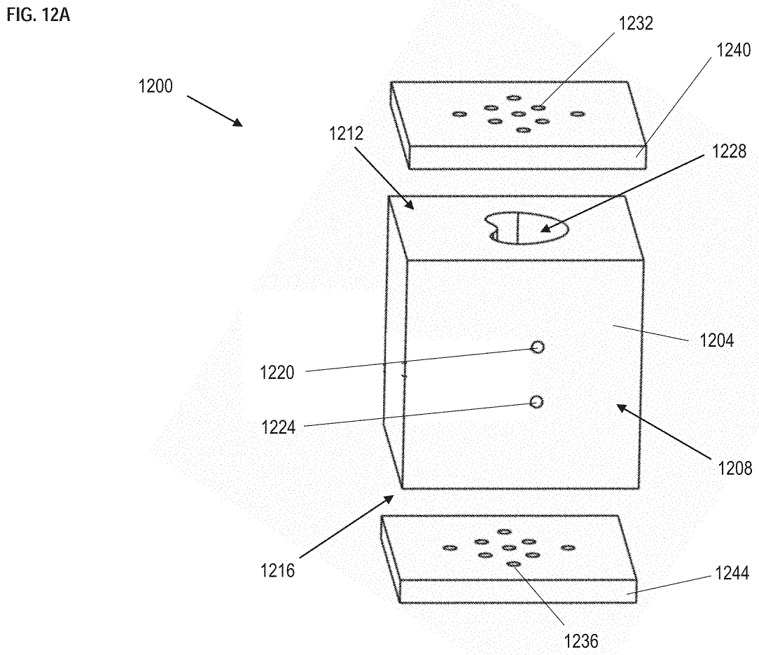

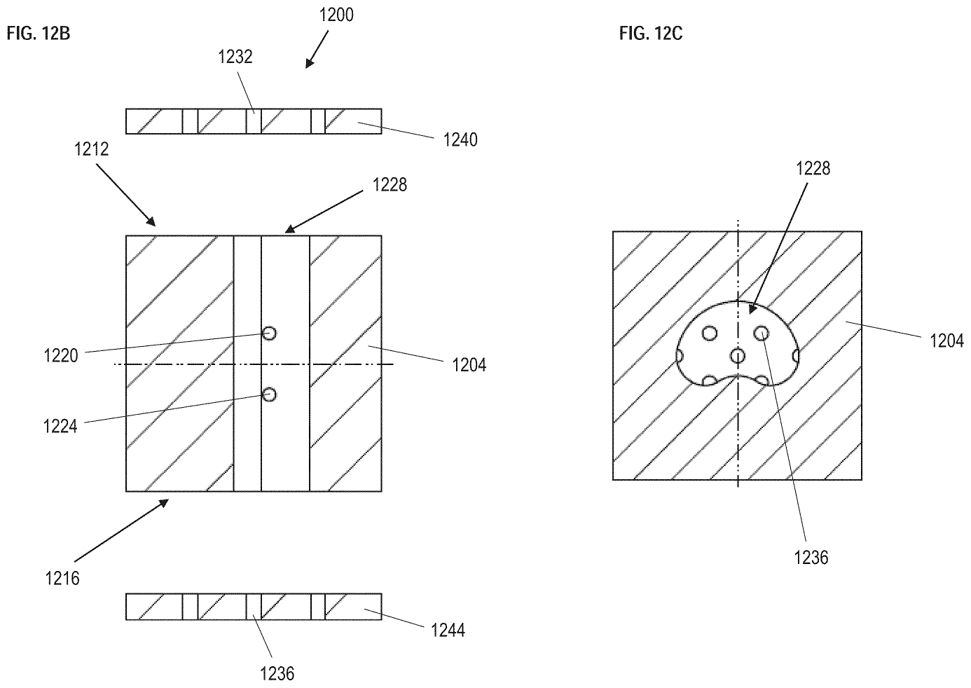

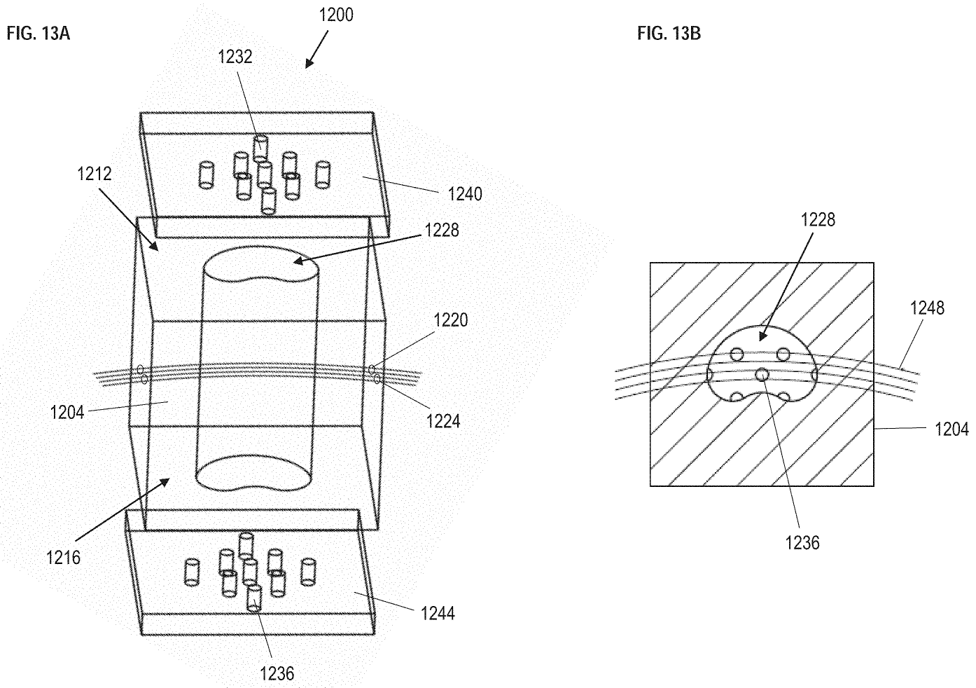

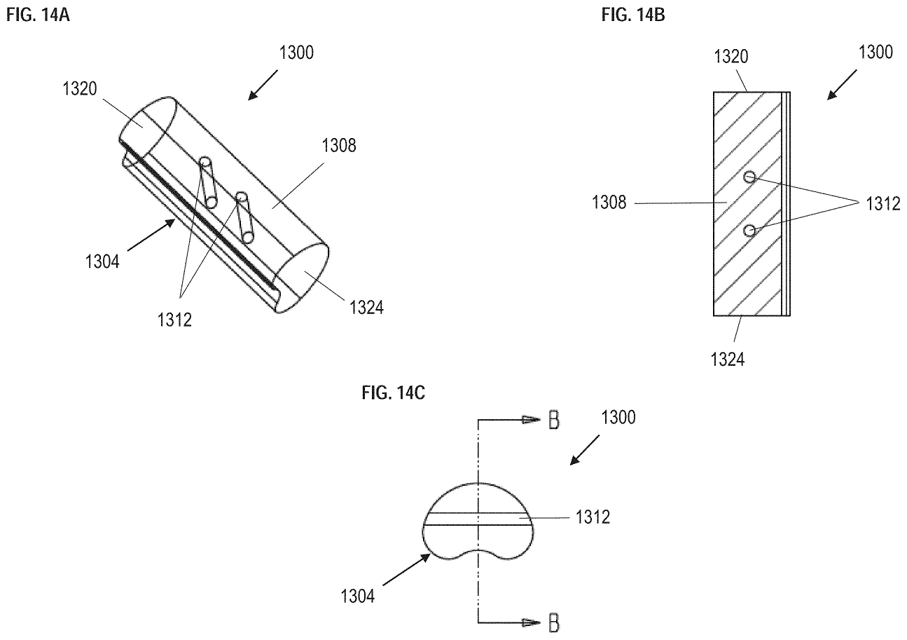

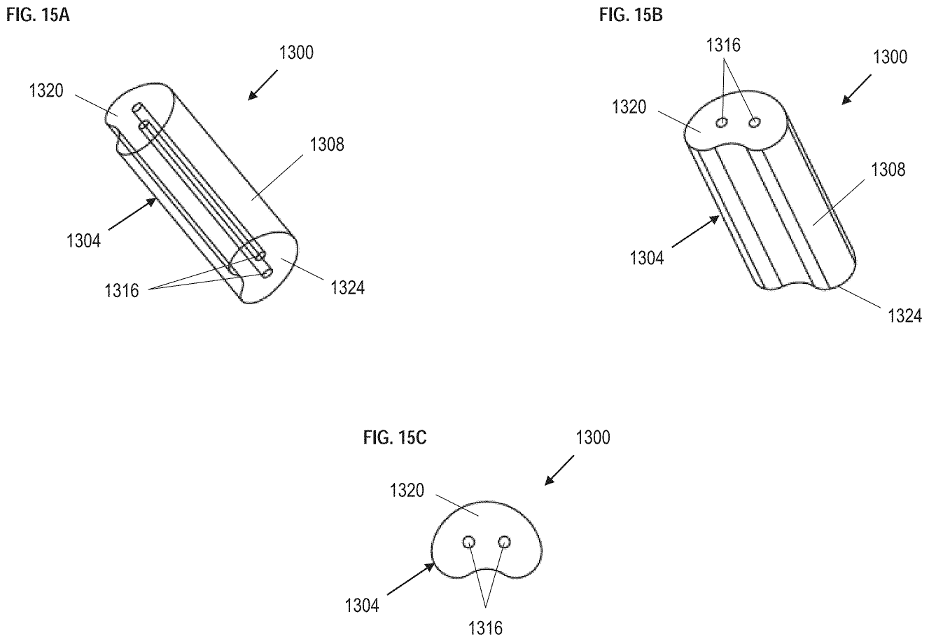

In a particular configuration, such as the one shown in FIGS. 12A-12C, and FIGS. 13A-13B, mold 1200 can be configured to form a lyophilized soft-tissue allograft 1300 where the periphery 1304 of the allograft body 1308 corresponds to an articular surface geometry of a patient's joint, and where the allograft body 1308 defines a plurality of suture passages 1312 extending either transversely through the body 1308, as best shown in FIGS. 14A-14C, or where the body 1308 defines a plurality of suture passages 1316 extending longitudinally through the first end 1320 and the second end 1324, as best shown in FIGS. 15A-15C. In this configuration, as best shown in FIGS. 12A-12B, and FIGS. 13A-13B, the body 1204 of mold 1200 has a peripheral surface 1208 extending between the first side 1212 and the second side 1216, and defines one or more suture passages (e.g., 1220, 1224) extending through the peripheral surface 1208 and into the chamber 1228. In some configurations, the one or more suture passages (e.g., 1220, 1224) of the body 1204 can extend through the peripheral surface 1208 and into the chamber 1228 in any or all three planes and/or longitudinally through the plurality of holes (1232, 1236) in the first end cap 1240 and the second end cap 1244. In this way, the suture passages (e.g., 1220, 1224) allow the lyophilized soft-tissue allograft 1300 to accept suture, wire, or other fixation components, and/or allow the user to create pilot holes in the allograft. In some configurations, such as the one shown in FIGS. 13A-13B, the suture passages (e.g., 1220, 1224) allow placement of sutures 1248 or other fixation components between multiple laminar layers of allograft tissue.

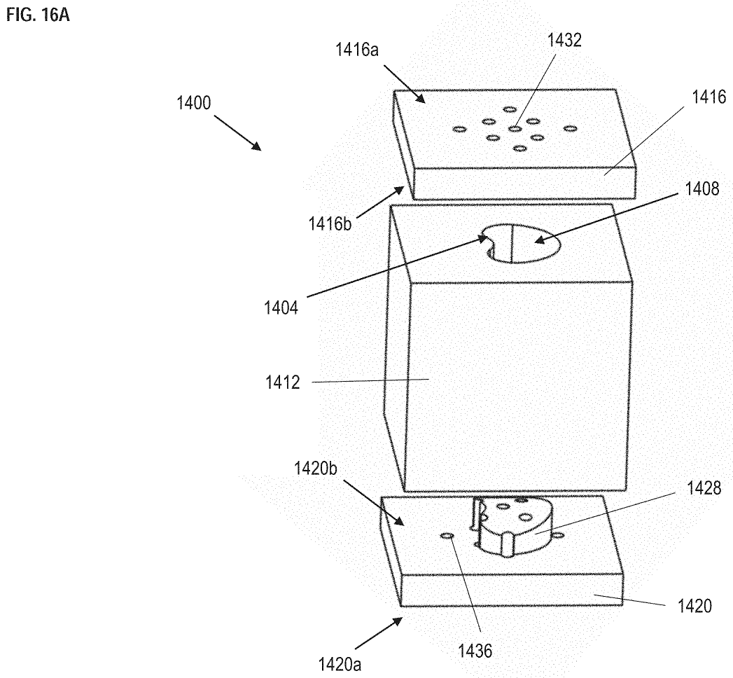

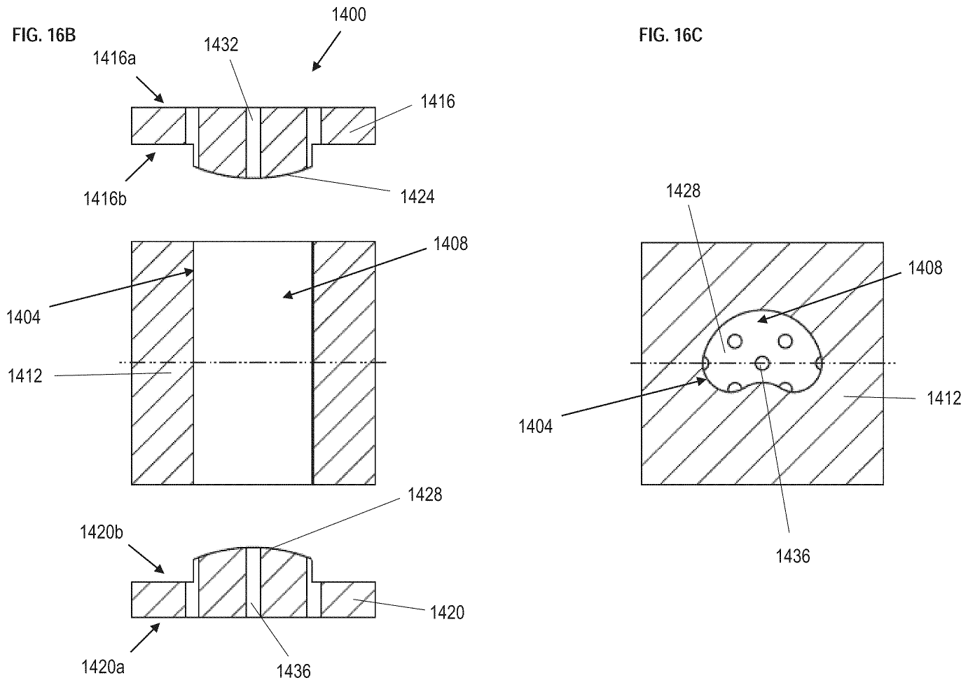

In a particular configuration, such as the one shown in FIGS. 16A-16C, mold 1400 can be configured to form a lyophilized soft-tissue allograft similar to allograft 1100, but with concave first and second ends, convex first and second ends, a concave first end and a convex second end, or a convex first end and a concave second end. In this configuration, as best shown in FIGS. 16A, 16C, the periphery 1404 of the chamber 1408 of the body 1412 corresponds to an articular surface geometry of a patient's joint. The first and second end caps (1416, 1420 respectively) may be configured to have a convex or concave surface portion that corresponds to an articular surface geometry of the patient's joint. As shown in FIG. 16B, the first end cap 1416 and second end cap 1420 each have a convex surface portion (1424, 1428 respectively), such that the lyophilized soft-tissue allograft formed in the chamber 1408 will have concave ends, and a plurality of holes (1432, 1436) extending between and through the outer sides (1416a, 1420a) and the body-facing sides (1416b, 1420b) of the first and second end caps (1416, 1420) and in fluid communication with the surface portions (1424, 1428) of the first end cap 1416 and the second end cap 1420 to aid in lyophilization.

In some configurations, the mold (e.g., 1000, 1200, 1400) can be configured to form a lyophilized soft-tissue allograft 1500, where the body 1504 of allograft 1500 has a cross-sectional shape having a convex portion 1508 and a concave portion 1512. In some configurations, the cross-sectional shape has a first curved portion 1516 with a first radius (R₁) 1520, and second curved portion 1524 with a second radius (R₂) 1528 that is larger than the first radius (R₁) 1520.

In some configurations of the present dermal allografts, dermal allograft 200 comprises a body having a first end and a second end, the allograft comprising lyophilized and compressed tissue layers between the first end and the second end, where the body has a periphery corresponding to the shape of the chamber of any of the presently disclosed molds.

In some configurations, each of the first and second ends 204, 208 of dermal allograft 200 is concave, as shown in FIGS. 2A-2B.

In some configurations, each of the first and second ends 404, 408 of dermal allograft 400 is convex, as shown in FIGS. 4A-4B.

In some configurations, the first end 504 of dermal allograft 500 is convex and the second end 508 of dermal allograft 500 is concave, as shown in FIGS. 5A-5B, or in some configurations, the first end 504 of dermal allograft 500 is concave and the second end 508 of dermal allograft is convex.

In some configurations, the peripheral surface (e.g., 212, 412, 512, 708, 908) of dermal allograft (e.g., 200, 400, 500, 700, 900) is cylindrical and has a circular cross-sectional shape, as shown in FIGS. 2A-2B, 4A-4B, 5A-5B, 7A-7B, and 9A-9B.

In some configurations, the body 712 of dermal allograft 700 comprises a plurality of longitudinal splines 704 spaced around the periphery 708 of the body 712, as shown in FIGS. 7A-7B.

In some configurations, the body 912 of dermal allograft 900 comprises a plurality of annular splines 904 around and spaced longitudinally along the periphery 908 of the body 912, as shown in FIGS. 9A-9B. In some configurations, the plurality of splines can be one or more helical splines and/or threads around and spaced longitudinally along the periphery 908 of the body 912.

In some configurations, the periphery 1104 of the body 1108 of dermal allograft 1100 corresponds to an articular surface geometry of a patient's joint, as shown in FIGS. 11A-11D.

In some configurations, the body 1308 of dermal allograft 1300 defines a plurality of suture passages 1312 extending transversely through the body 1308, as shown in FIGS. 14A-14C.

In some configurations, the body 1308 of dermal allograft 1300 defines a plurality of suture passages 1316 extending longitudinally through the first end 1320 and the second end 1324, as shown in FIGS. 15A-15C.

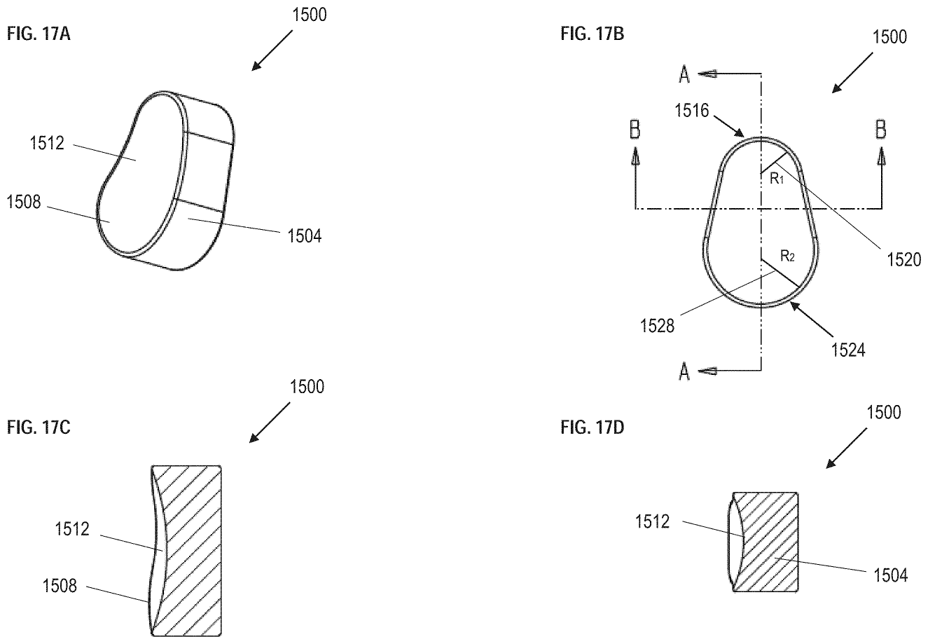

In some configurations, the body 1504 of dermal allograft 1500 has a cross-sectional shape having a convex portion 1508 and a concave portion 1512, as shown in FIGS. 17A-17D.

In some configurations, the cross-sectional shape of the body 1504 of dermal allograft 1500 has a first curved portion 1516 with a first radius 1520, and second curved portion 1524 with a second radius 1528 that is larger than the first radius 1520, as shown in FIG. 17B.

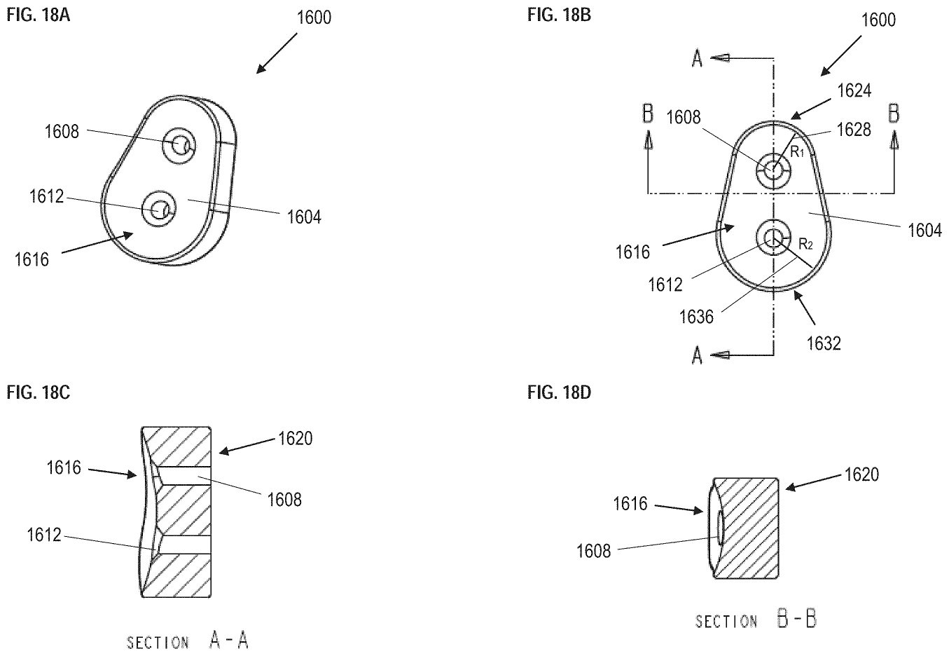

In some configurations, the body 1604 of dermal allograft 1600 defines one or more channels (e.g., 1608, 1612) extending between and through the first end 1616 and second end 1620, as shown in FIGS. 18A-18D. In some configurations, the cross-sectional shape of the body 1604 of dermal allograft 1600 has a first curved portion 1624 with a first radius 1628, and a second curved portion 1632 with a second radius 1636 that is larger than the first radius 1628, as shown in FIG. 18B.

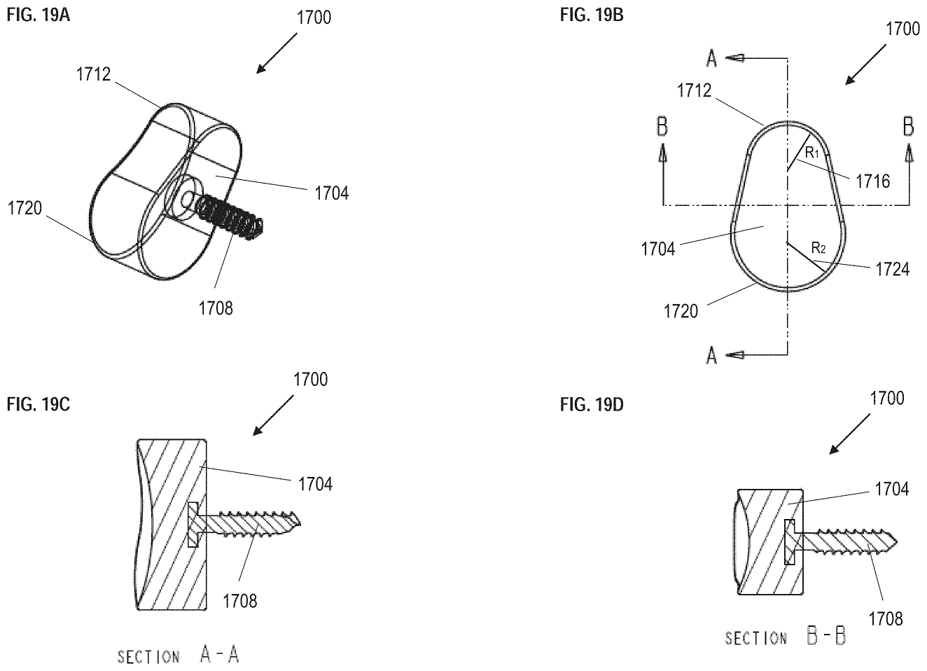

In some configurations, a fixation component 1708 (e.g., suture anchor, bone screw) is embedded in the body 1704 of dermal allograft 1700, which is shaped similarly to dermal allografts 1500, 1600 with the cross-sectional shape of the body 1704 having a first curved portion 1712 with a first radius 1716, and a second curved portion 1720 with a second radius 1724 that is larger than the first radius 1716, as shown in FIG. 19B. In this way, when fixation component 1708 is embedded into the body 1704 of dermal allograft 1700, a stronger interface between the fixation component 1708 and body 1704 is created, as compared to inserting a fixation component into the dermal allograft after the allograft is formed. Additionally, in this way, the surgical procedure is easier to complete for the surgeon as it reduces surgical time, provides for a more accurate placement of the entire dermal allograft as the relative position of the fixation component to the allograft is fixed, and can also make it easier for accurate preparation of the surgical site with precision instruments that are matched with the designs and dimensions of the dermal allograft.

In some configurations, the fixation component 1708 embedded in the body 1704 of dermal allograft 1700 is a suture anchor.

In some configurations, the fixation component 1708 embedded in the body 1704 of dermal allograft 1700 is a bone screw.

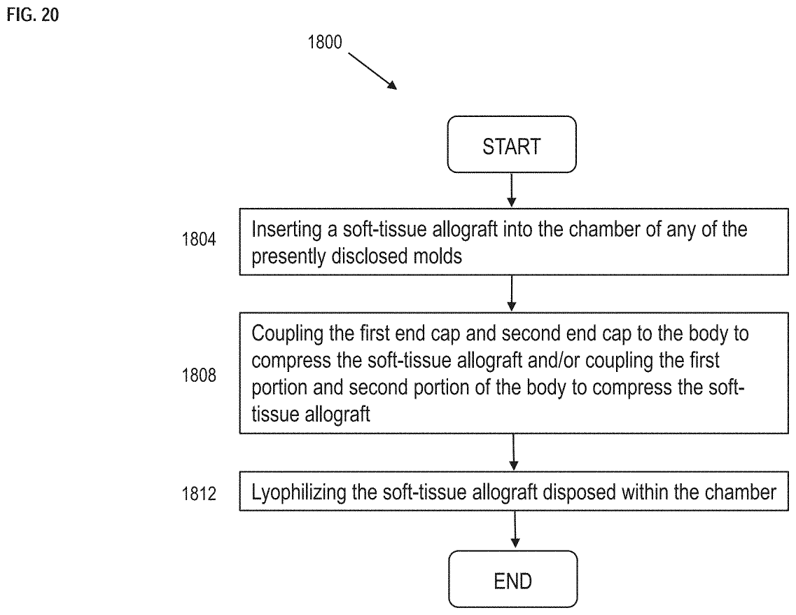

Referring now to FIG. 20, a flow chart showing a method 1800 for forming a lyophilized soft-tissue allograft using any of the configurations of the molds of the present disclosure is shown. The operation of method 1800 will be described with reference to mold 100, but may be performed with any of the configurations of the present molds of the present disclosure. In some implementations of the present methods, a lyophilized soft-tissue allograft is formed by (a) inserting a soft-tissue allograft into the chamber of any of the presently disclosed molds; (b) coupling the first end cap and second end cap to the body to compress the soft-tissue allograft; and (c) lyophilizing the soft-tissue allograft disposed within the chamber.

At block 1804, and as illustrated by FIGS. 1A-2C, method 1800 starts by inserting a soft-tissue allograft (e.g., 120) into the chamber (e.g., 116) of any of the presently disclosed molds. At block 1808, method 1800 continues by coupling the first end cap (e.g., 128) and second end cap (e.g., 144) to the body (e.g., 104) to compress the soft-tissue allograft (e.g., 120). At block 616, method 1800 continues by lyophilizing the soft-tissue allograft (e.g., 120) disposed within the chamber (e.g., 116).

In some implementations of method 1800, the soft-tissue allograft (e.g., 120) may be lyophilized one or more times prior to insertion into the chamber (e.g., 116). In some implementations of method 1800, the soft-tissue allograft (e.g., 120) may be lyophilized anywhere from 10% to 90% prior to insertion into the chamber (e.g., 116) for further lyophilization. In some implementations of method 1800, the remaining lyophilization of the soft-tissue allograft takes place inside the chamber.

The above specification and examples provide a complete description of the structure and use of exemplary configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the present devices, apparatuses, kits, and methods are not intended to be limited to the particular forms disclosed. Rather, they include all modifications and alternatives falling within the scope of the claims, and configurations other than the one shown may include some or all of the features of the depicted configuration. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one configuration or may relate to several configurations.

The claims are not intended to include, and should not be interpreted to include, means-plus- or step-plus-function limitations, unless such a limitation is explicitly recited in a given claim using the phrase(s) “means for” or “step for,” respectively.

1. A mold for forming lyophilized allograft implants, comprising:
a body having a first side and a second side and defining a chamber for receiving an allograft tissue, the chamber having a periphery extending between and through the first side and the second side;
a first end cap configured to be coupled to the first side of the body over the chamber, the first end cap having a body-facing side with a surface portion that is aligned with the chamber when the first end cap is coupled to the body, and an outer side defining a plurality of holes extending between and through the outer side and the body-facing side and in fluid communication with the surface portion of the first end cap; and
a second end cap configured to be coupled to the second side of the body over the chamber, the second end cap having a body-facing side with a surface portion that is aligned with the chamber when the second end cap is coupled to the body, and an outer side defining a plurality of holes extending between and through the outer side and the body-facing side and in fluid communication with the surface portion of the second end cap.

2. The mold of claim 1, where the body, having a first portion and a second portion, defines the chamber when the first portion and the second portion are coupled together.

3. The mold of claim 1, where the first end cap and the second end cap are coupled to the body to compress and form the allograft tissue into a shape conforming to the chamber during lyophilization, and/or the first portion and the second portion of the body are coupled to compress and form the allograft tissue into a shape conforming to the chamber during lyophilization.

4. The mold of claim 1, where the chamber is cylindrical.

5. The mold of claim 1, where the surface portion of the first end cap is convex and the surface portion of the second end cap is convex.

6. The mold of claim 1, where the surface portion of the first end cap is concave and the surface portion of the second end cap is concave.

7. The mold of claim 1, where the surface portion of the first end cap is convex and the surface portion of the second end cap is concave.

8. The mold of claim 1, where the surface portion of the first end cap is concave and the surface portion of the second end cap is convex.

9-14. (canceled)

15. The mold of claim 1, where the body has a peripheral surface extending between the first side and the second side, and defining one or more suture passages extending through the peripheral surface and into the chamber.

16. A dermal allograft comprising:
a body having a first end and a second end, the allograft comprising lyophilized and compressed tissue layers between the first end and the second end, where the body has a periphery corresponding to the shape of the chamber of the mold of claim 1.

17. The dermal allograft of claim 16, where each of the first and second ends is concave.

18. The dermal allograft of claim 16, where each of the first and second ends is convex.

19. The dermal allograft of claim 16, where the first end is convex and the second end is concave.

20. The dermal allograft of claim 16, where the first end is concave and the second end is convex.

21. The dermal allograft of claim 16, where the peripheral surface is cylindrical and has a circular cross-sectional shape.

22-25. (canceled)

26. The dermal allograft of claim 16, where the body defines a plurality of suture passages extending transversely through the body.

27. The dermal allograft of claim 16, where the body defines a plurality of suture passages extending longitudinally through the first end and the second end.

28-29. (canceled)

30. The dermal allograft of claim 16, where the body defines one or more channels extending between and through the first end and second end.

31-33. (canceled)

34. A method comprising:
(a) inserting a soft-tissue allograft into the chamber of the mold of claim 1;
(b) coupling the first end cap and second end cap to the body to compress the soft-tissue allograft and/or coupling the first portion and second portion of the body to compress the soft-tissue allograft; and
(c) lyophilizing the soft-tissue allograft disposed within the chamber.

35. The method of claim 34, where the soft-tissue allograft is lyophilized anywhere from 10% to 90% prior to inserting into the chamber of the mold of claim 1.
